SEQUENCE LISTING (2 sequences)

SEQ ID NO: 1 - DNA, 1745 bp, Homo sapiens
agcctgacttcagcgctcccactctcggccgacacccctcatggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtgacaaagtgactgcccggtcagatcagctggatctcagcggggagccacgtctcttgcactgtggtctctgcatggaccccagggctgtggggacttgggggacagtaatcaagtaatccccttttccagaatgcattaacccactcccctgacctcacgctggggcaggtccccaagtgtgcaagctcagtattcatgatggtgggggatggagtgtcttccgaggttcttgggggaaaaaaaattgtagcatatttaagggaggcaatgaaccctctcccccacctcttccctgcccaaatctgtctcctagaatcttatgtgctgtgaataataggccttcactgcccctccagtttttatagacctgaggttccagtgtctcctggtaactggaacctctcctgagggggaatcctggtgctcaaattaccctccaaaagcaagtagccaaagccgttgccaaaccccacccataaatcaatgggccctttatttatgacgactttatttattctaatatgattttatagtatttatatatattgggtcgtctgcttcccttgtatttttcttcctttttttgtaatattgaaaacgacgatataattattataagtagactataatatatttagtaatatatattattaccttaaaagtctatttttgtgttttgggcatttttaaataaacaatctgagtgt

SEQ ID NO: 2 - protein, 326 aa, Homo sapiens
MDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSE

(19) United States
(12) Patent Application Publication
(10) Pub. No.: US 2010/0055705 A1
(43) Pub. Date: Mar. 4, 2010

(54) COMPOSITIONS AND METHODS FOR DIAGNOSING AND TREATING CANCER

(76) Inventors: Gerald M. Wilson, Middle River, MD (US); Sarah Brennan, Baltimore, MD (US); Nadim Alkharouf, Abingdon, MD (US); Yuki Kuwano, Baltimore, MD (US); Perry Blackshear, Chapel Hill, NC (US); Myriam Gorospe, Baltimore, MD (US)

Correspondence Address: MORGAN LEWIS & BOCKIUS LLP, 1111 PENNSYLVANIA AVENUE NW, WASHINGTON, DC, 20004, US

(21) Appl. No.: 12/550,601

(22) Filed: Aug. 31, 2009

Related U.S. Application Data
(60) Provisional application No. 61/093,003, filed on Aug. 29, 2008.

(51) Int. Cl. C12Q 1/68; C12Q 1/02

(52) U.S. Cl. 435/6; 435/29

(57) ABSTRACT

The invention is drawn novel methods and compositions for the treatment of cancer, and for the diagnosis and prognosis of cancer in a subject. In particular aspects, the invention relates to the finding that the protein tristetraprolin (TTP) is decreased or repressed in a myriad and diversity of cancers. To this end, TTP represents a viable therapeutic option for the treatment of cancer. Additionally, TTP represents a clinically useful biomarker for the diagnosis and prognosis of cancer.

### CROSS-REFERENCE TO RELATED APPLICATIONS

The application claims priority to U.S. Provisional Application No. 61/093,003, filed 29 Aug. 2008, which is incorporated by reference in its entirety.

### STATEMENT REGARDING FEDERALLY SPONSORED RESEARCH OR DEVELOPMENT

Part of the work performed during development of this invention utilized U.S. Government funds under National Institutes of Health Grant No. CA102428. The U.S. Government has certain rights in this invention.

### SEQUENCE LISTING SUBMISSION VIA EFS-WEB

A computer readable text file, entitled “100413-5028-SequenceListing.txt,” created on or about Aug. 31, 2009 with a file size of about 6 kb contains the sequence listing for this application and is hereby incorporated by reference in its entirety.

### BACKGROUND OF THE INVENTION

1. Field of the Invention

The invention relates to cancer biology. The invention further relates to compositions and methods for treating and/or diagnosing cancer.

2. Background of the Invention

AU-rich elements (AREs) are potent cis-acting determinants of mRNA decay and translational efficiency that function through interactions with diverse trans-acting factors, collectively termed ARE-binding proteins (ARE-BPs). Transcripts targeted by these proteins often encode factors that directly impact critical processes such as cell division, apoptosis, angiogenesis, and inflammation, which all play a role in oncogenesis and tumor progression. The inventors of the present invention have found that the altered expression of one or more ARE-BP and/or an mRNA transcript that binds to an ARE-BP and/or a corresponding encoded protein of an mRNA transcript that binds to an ARE-BP represents a viable therapeutic option for the treatment of cancer and/or use in methods for the diagnosis and prognosis of cancer. In particular aspects, the ARE-BP tristetraprolin (TTP) was found to be a viable therapeutic option for the treatment of cancer, and in uses for methods for the diagnosis and prognosis of cancer, including, for example, cancers that are particularly aggressive.

### SUMMARY OF THE INVENTION

The present invention provides methods of assessing the risk of a subject having an abnormal condition, such as cancer. The methods of the present invention comprise determining levels of tristetraprolin (TTP) in a sample suspected of being abnormal in a subject and comparing the levels of TTP in the sample from the subject to normal levels of TTP. In one embodiment, lower levels of TTP, compared to normal levels of TTP, indicate that the subject has an increased risk of having the abnormal condition. The present invention also provides kits for performing these methods.

The present invention also provides methods of assessing the progression of an abnormal condition, such as cancer in a subject having the abnormal condition. The methods comprise determining levels of TTP in a sample that is abnormal in the subject at a first and second time point and comparing the levels of TTP from the first and second time points to determine a change in the levels of TTP over time. In one embodiment, increased levels of TTP over time indicates that the abnormal condition in the subject may be regressing, whereas decreased levels of TTP over time indicates that the abnormal condition in the subject may be progressing. The present invention also provides kits for performing these methods.

The present invention also relates to methods of increasing the levels of TTP in a cell. In one embodiment, the methods comprise comprising introducing into the cell a vector encoding a TTP protein. In another embodiment, the methods comprise administering to the cell a histone deacetyltransferase (HDAC) inhibitor.

The present invention also relates to methods of assessing the risk of a subject having cancer, the method comprising determining levels of TTP in a sample suspected of being cancerous in the subject, and comparing the levels of TTP in the sample from the subject to normal levels of TTP. Lower levels of TTP compared to normal levels of TTP would indicate that the subject has an increased risk of having cancer.

The present invention also relates to methods of increasing the levels of TTP in a cell comprising introducing into the cell a vector, the vector comprising a polynucleotide encoding a TTP protein, the protein comprising residues 2-326 of SEQ ID NO:2.

The present invention also relates to methods of increasing the levels of TTP in a cell comprising administering to the cell a histone deacetyltransferase (HDAC) inhibitor and determining the levels of TTP in a cell after administration of the HDAC inhibitor.

### BRIEF DESCRIPTION OF THE DRAWINGS

FIG. 1 depicts the expression of ARE-BP mRNAs in tumors versus peripheral non-transformed tissues. A cDNA Cancer Profiling Array was probed for expression of selected ARE-BP mRNAs. (A) Array hybridization signals from lung, breast, and cervical cDNA samples probed for TTP and ubiquitin (ub) expression in both tumors (T) and patient-matched, non-transformed peripheral tissue (N). (B) Scatter plots showing ubiquitin-normalized ratios of AUF1, TIA-1, TTP, and HuR cDNAs derived from tumors versus patient-matched normal tissues. Solid lines (at ratio=1) indicate equivalent ARE-BP expression in tumors and peripheral non-transformed tissues. A difference of one log2 unit (100% increase or 50% decrease; dotted lines) in ARE-BP expression between tumor and normal tissues was considered substantial. n indicates the number of matched patient sample pairs for each tissue type (bottom). Asterisks in TTP panels denote tissues where TTP expression was undetectable above background in selected tumors (3 testicular tumors, 1 skin tumor).

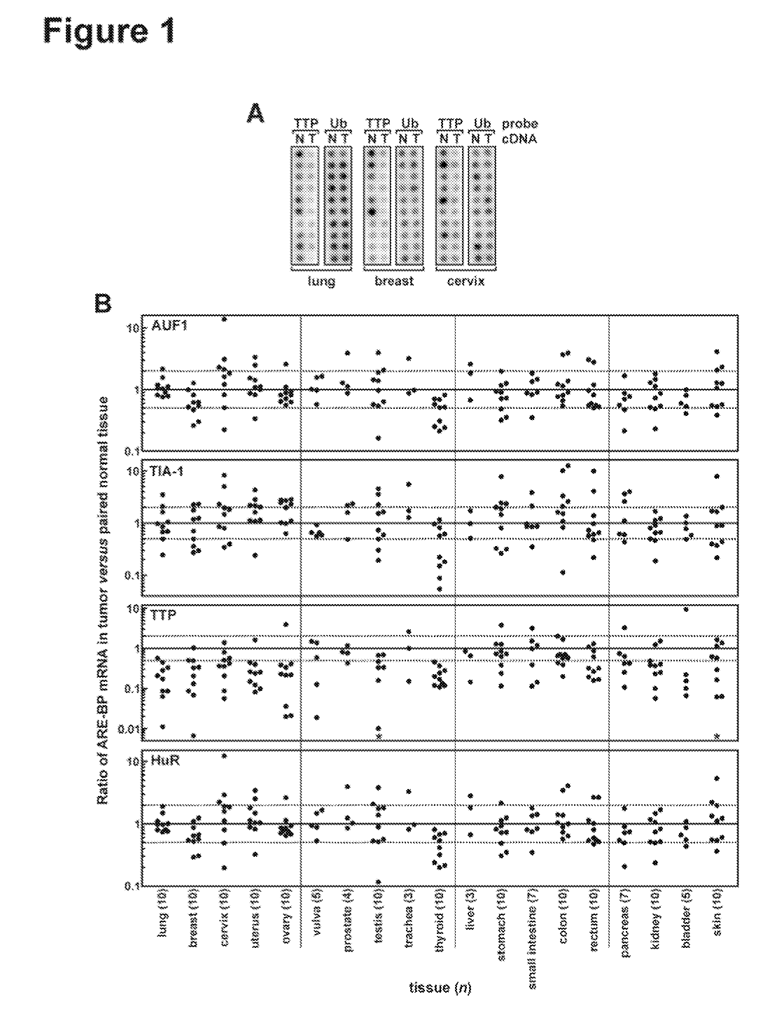

FIG. 2 depicts the repression of TTP expression in cancer cell lines and human tumors. (A) Expression of TTP mRNA was measured in nine human cancer cell lines concomitantly with human tissue samples on the cDNA Cancer Profiling Array. Bars labeled lung, breast, and cervix each represent the mean TTP hybridization signals (±SD) from ten non-transformed tissues, all normalized to ubiquitin expression. (B) Western blots measuring TTP and β-actin protein levels in whole cell extracts from breast tumors (T) and patient-matched peripheral non-transformed tissue (N) from five patients. (C) Gene array datasets were screened for differential TTP mRNA levels using the Oncomine v3.0 utility. Median TTP mRNA levels are shown by solid lines within each box on distribution plots. Upper and lower limits of each box represent the 75th and 25th percentiles, respectively, while the extended lines indicate 10th and 90th percentiles for each dataset.

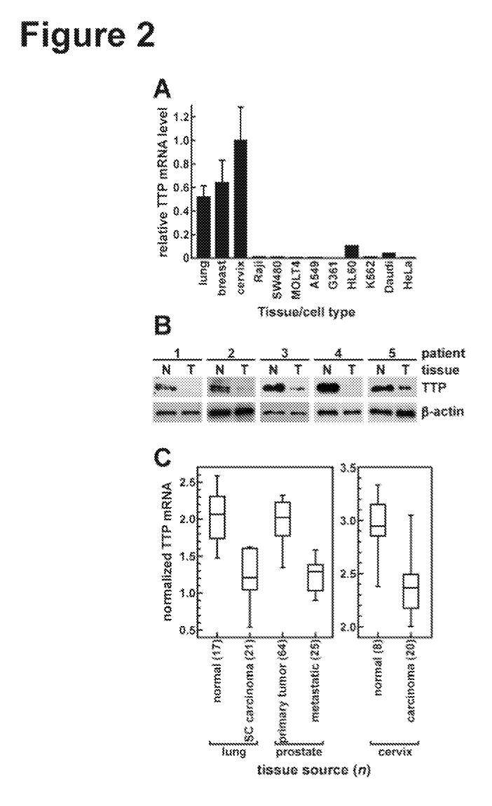

FIG. 3 depicts the influence of TTP on tumor cell phenotypes. (A) Western blots targeting the FLAG epitope show levels of FLAG-TTPwt and FLAG-TTP C 147R in stably transfected HeLa/Tet-Off cultures 24 hours following removal of doxycycline (Dox) (upper panel). Expression from FLAG-TTP transgenes was also compared to endogenous TTP protein levels in a cervical tissue lysate (CTL) by Western blot using anti-TTP antibodies (bottom panel). (B) Phase contrast photomicrographs showing morphological features of HeLa cells before doxycycline (+Dox) and 24 hours after doxycycline (−Dox) activation of FLAG-TTPwt and C147R transgenes. (C) Proliferation of untransfected HeLa cells (ut, closed circles) or cells expressing wild type (open circles) or C 147R mutant (triangles) forms of FLAG-TTP was measured. Each point represents the mean±SD of at least five measured cell populations. Triplicate independent experiments yielded similar results. (D) Untransfected or TTP wt/C147R-expressing HeLa cells were counted 24 hours following treatment with various concentrations of staurosporine and cisplatin. Symbol assignments are identical to (C) and represent the mean±SD of at least four cell populations.

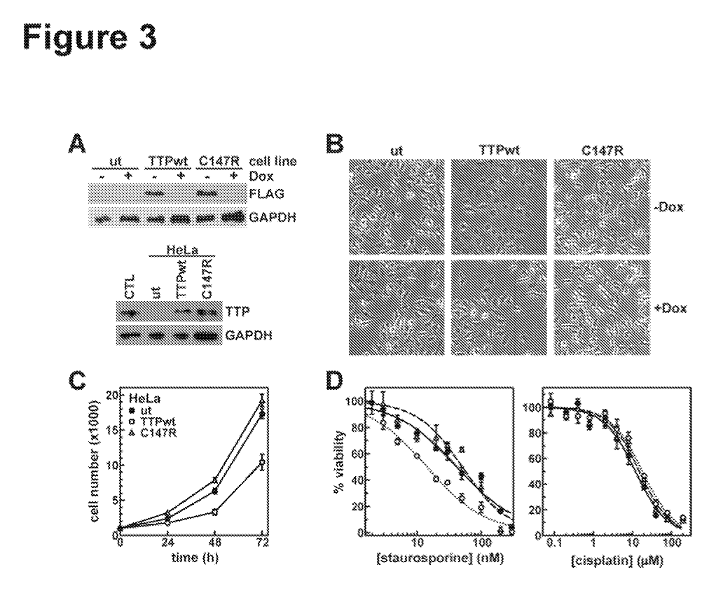

FIG. 4 depicts the restoration of cellular TTP suppressing the expression of the pro-angiogenic factor vascular endothelial growth factor (VEGF) in HeLa cells by destabilization of its mRNA. (A) Relative VEGF mRNA levels were measured by qRT-PCR in untransfected HeLa cells (ut) or cells stably transfected with wild type (TTPwt) or mutant (C147R) TTP prior to doxycycline (+Dox) and 24 hours following doxycycline (−Dox) induction of FLAG-TTPwt or C147R expression. Each bar represents the mean±SD of three independent samples. (B) Representative actinomycin D time course experiments showing the decay kinetics of VEGF mRNA in untransfected HeLa cells (closed circles), and cells expressing wild type (open circles) or C147R mutant (triangles) forms of FLAG-TTP. Lines indicate regression solutions to a single exponential decay model. VEGF mRNA half-life values resolved from replicate independent experiments are given in the text. (C) Ribonucleoprotein immunoprecipitation experiments were performed using control IgG or anti-FLAG antibodies and cell lysates from indicated HeLa cell lines. Each immunoprecipitate was screened for VEGF and GAPDH mRNAs by qualitative RT-PCR. (D) VEGF mRNA levels were also measured in anti-FLAG immunoprecipitates by quantitative real-time PCR, and are shown as the mean±SD of three qPCR reactions normalized to GAPDH mRNA levels. An independent replicate experiment yielded similar results.

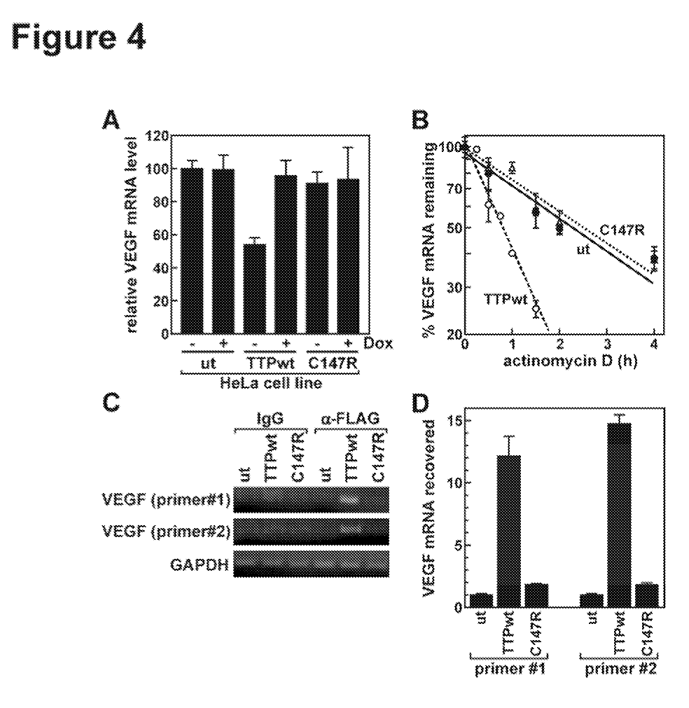

FIG. 5 depicts the correlation analyses of TTP expression versus pathological features and clinical outcomes in breast cancer. Relative TTP mRNA levels were extracted from an array dataset containing gene expression profiles for 249 human breast tumors. Construction of this dataset (GEO Acc# GSE3494) is described in Proc. Natl. Acad. Sci. USA 2005 Sep. 20; 102(38):13550-5 (incorporated by reference), and includes the Elston-Ellis pathological grade of each tumor at excision and patient mortality from recurrent breast cancer over the subsequent 13 years. (A) A negative correlation between TTP expression and breast tumor grade (r=−0.431, P=1.1×10-12) was resolved using Oncomine v3.0. Distribution plots were assembled as described in FIG. 2, with the total number of tumors in each grade pool indicated by n. (B) vascular endothelial growth factor (VEGF) mRNA levels are negatively correlated with TTP mRNA in breast tumors (r=−0.281, P=5.9×10-6). Black circles indicate grade I tumors, green circles grade II, red circles grade III, and open circles represent tumors of undefined pathological grade. Dotted lines indicate the 95% confidence intervals of the regression solution. (C) The patient pool was ranked by tumor TTP expression level and subdivided into thirds. Kaplan-Meier analysis of each cohort revealed that patients with the lowest tumor TTP mRNA levels (red line) experienced a significant increase (P=0.01) in the risk of death from recurrent breast cancer relative to patients expressing the highest levels of TTP mRNA (black line). Patients in the middle cohort (green line) were also at higher risk for death from recurrent cancer than patients ranked in the top third (P=0.04).

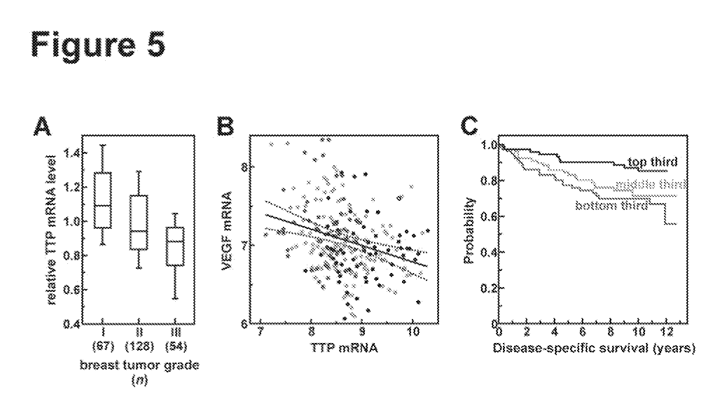

FIG. 6 depicts the distribution plots of TTP mRNA levels from additional gene array datasets that were constructed using the Oncomine v3.0 utility as described in FIG. 2. The number of patient samples analyzed (n) is indicated for each tissue pool.

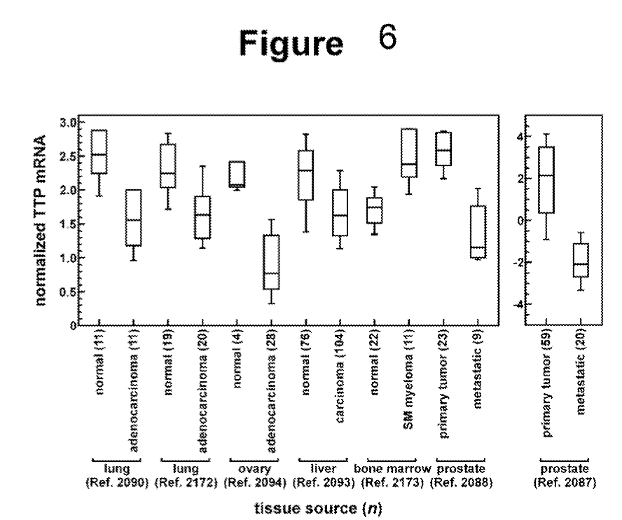

FIG. 7 depicts PARP cleavage that verifies that staurosporine-induced HeLa cell death is apoptotic. Staurosporine (100 nM) was added to cultures of untransfected and FLAG-TTP-expressing HeLa cells. At indicated time points, whole cell lysates were prepared and analyzed for PARP cleavage by Western blot. Bands corresponding to full length PARP (116 kDa) and the large caspase-3 cleavage product (89 kDa) are indicated.

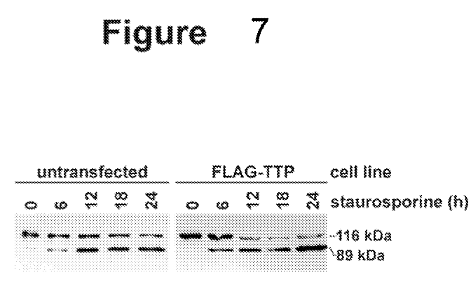

FIG. 8 depicts the influence of TTP on tumorigenic phenotypes in a non-transformed cell model. (A) Western blot showing TTP and GAPDH protein levels in MEFs from TTP knockout mice (−/−) and wild-type littermates (+/+). The minor band remaining in extracts from the TTP−/− MEFs likely represents a cross-reactive protein target, possibly a TTP family member. (B) Proliferation assays of TTP−/− and TTP+/+ MEFs performed as described in FIG. 3. (C) The sensitivity of TTP−/− (closed circles) and TTP+/+ (open circles) MEFs to pro-apoptotic stimuli staurosporine (left) and cisplatin (right) were assessed as described in FIG. 3. (D) Relative VEGF mRNA levels and (E) VEGF mRNA turnover rates in TTP−/− and TTP+/+ MEFs were measured as described in FIG. 4. Parameters describing VEGF mRNA decay kinetics in MEF models are given in the text.

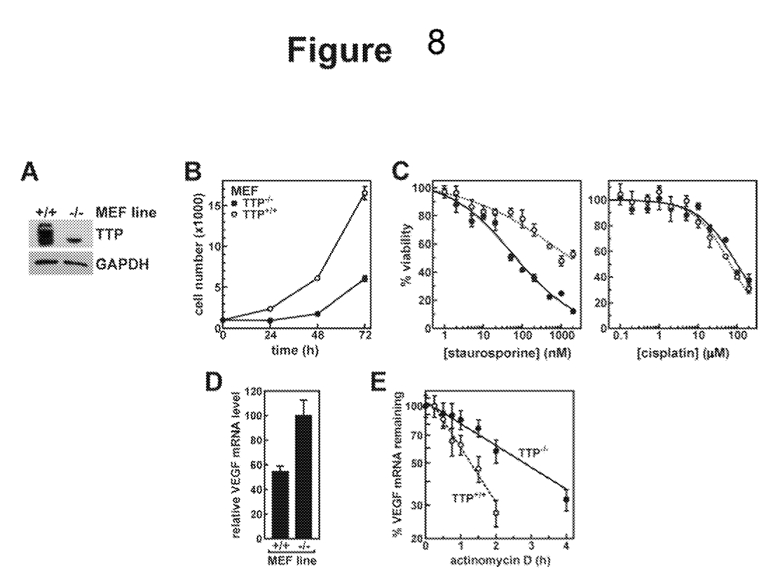

FIG. 9 depicts vascular endothelial growth factor (VEGF) mRNA levels not correlating with TTP expression in prostate cancer. Relative TTP and VEGF mRNA levels were extracted from an array dataset containing gene expression profiles for 89 primary (black circles) and metastatic (green circles) human prostate tumors. Correlation analysis of VEGF versus TTP mRNA levels demonstrated no relationship between these transcripts (r=−0.038; P=0.72). Dotted lines indicate the 95% confidence intervals of the regression solution.

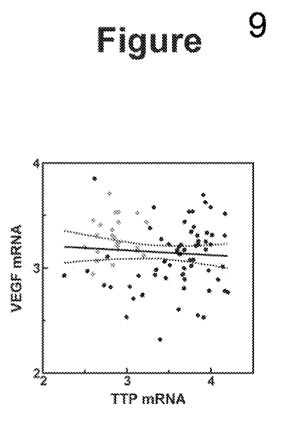

FIG. 10 depicts a schematic of how the repression of TTP expression may reprogram a post-transcriptional gene regulatory network in cancer.

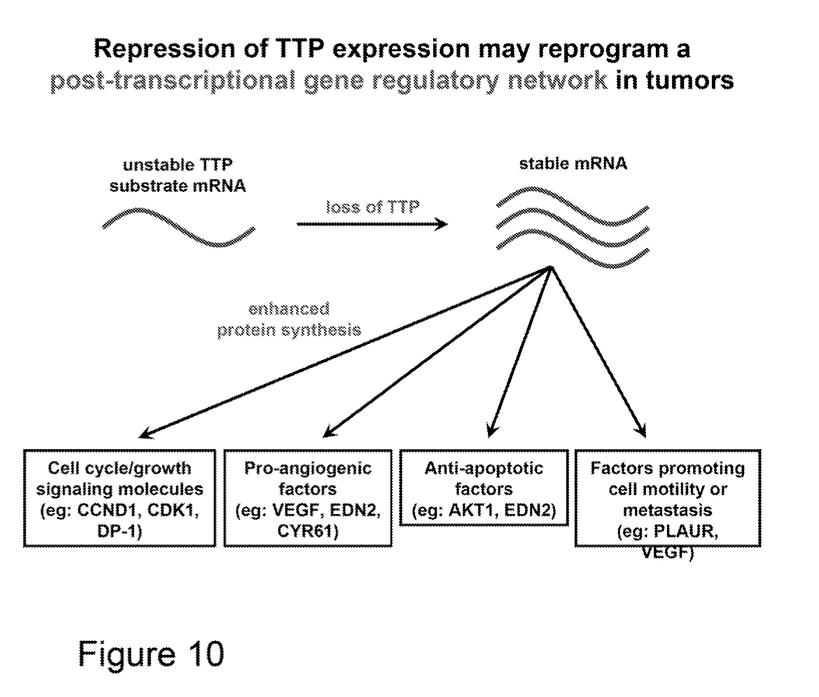

FIG. 11 depicts the TTP polynucleotide sequence.

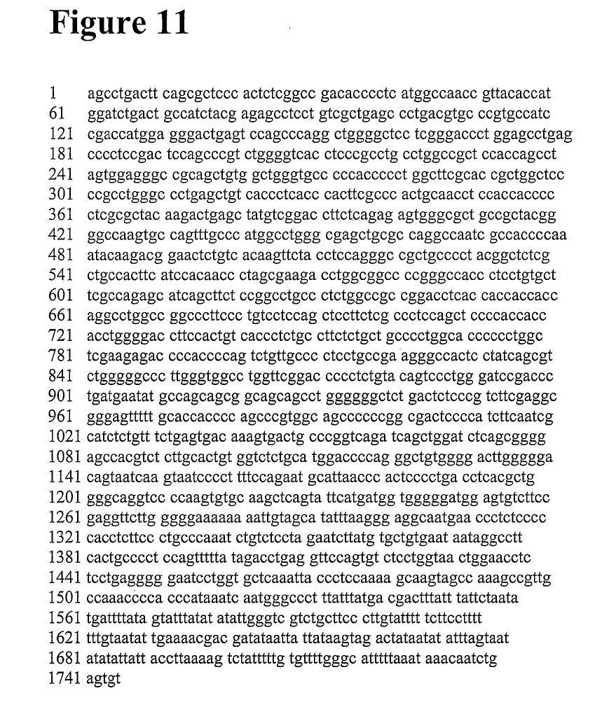

FIG. 12 depicts the TTP amino acid sequence.

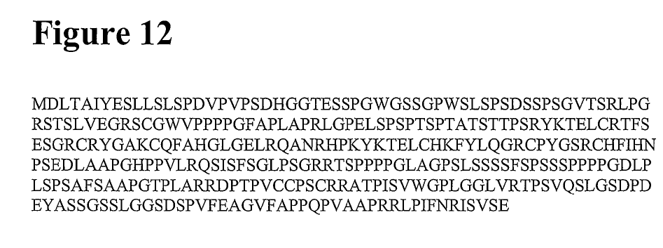

FIG. 13 depicts the effect of histone deacetyltransferase (HDAC) inhibitors on TTP protein expression in cell.

### DETAILED DESCRIPTION OF THE INVENTION

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341), all of which are incorporated by reference.

As used herein, “a” or “an” may mean one or more. As used herein when used in conjunction with the word “comprising”, the words “a” or “an” may mean one or more than one. As used herein “another” may mean at least a second or more. Furthermore, unless otherwise required by context, singular terms include pluralities and plural terms include the singular.

As used herein, “treat” and all its forms and tenses (including, for example, treating, treated, and treatment) can refer to therapeutic or prophylactic treatment. In certain aspects of the invention, those in need thereof of treatment include those already with a pathological condition of the invention (including, for example, a cancer), in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of skill in the art realizes that a treatment may improve the patient's condition, but may not be a complete cure of the pathological condition. In other certain aspects of the invention, those in need thereof of treatment include, those in which a pathological condition of the invention (including, for example, a cancer) is to be prevented, in which case treating refers to administering to a subject a therapeutically effective amount of a composition to a subject (including, for example, a human or other mammal in need of treatment) at risk of developing a pathological conditional of the invention.

The present inventors evaluated, inter alia, changes in the expression of four well characterized ARE-BPs (AUF1, TIA-1, HuR, and TTP) across a variety of human neoplasms using three principal methods: (i) cDNA arrays comparing expression in 154 tumors from 18 different tissue types versus patient-matched non-transformed tissues, (ii) meta-analyses of gene chip studies comparing expression in normal versus primary and metastatic tumors across diverse tissue types, and (iii) comparing EST and SAGE frequency between normal versus cancerous cells derived from many tissue sources. For three ARE-BPs surveyed; AUF1, TIA-1, and HuR, expression was not systematically dysregulated in cancers; however, in selected tissues, expression of some of these proteins was frequently up- or down-regulated to a significant extent. For example, HuR expression was dramatically increased in many leukemias, and moderately induced in most melanomas and bladder cancers. AUF1 expression increased or decreased in tumors depending on tissue type, including modest increases in AUF1 mRNA levels as a function of tumor grade in breast cancer. Based on the results for AUF1, TIA-1, and HuR, the present inventors did not expect to see that TTP would be systematically repressed. To this end, it was surprisingly found that the expression of TTP was significantly decreased in a plethora and diversity of tumor types, and was robustly repressed in aggressive cancers of the breast and prostate. These data provide support that dysregulated expression of one or more ARE-BP and/or an mRNA transcript that binds to an ARE-BP and/or a corresponding encoded protein of an mRNA transcript that binds to an ARE-BP may contribute to oncogenesis or tumor progression and thus represent a therapeutic target for the treatment, diagnosis, and/or prognosis of cancer.

Bioinformatic analyses of gene chip was also carried out and demonstrated that TTP mRNA varies inversely with tumor grade in breast cancer, and that loss of TTP expression correlates with increased VEGF mRNA in these patients. By contrast, comparable studies of prostate cancer patients show significant repression of TTP expression between primary and metastatic tumors, but no correlation with VEGF expression. Together, these data indicate that repression of TTP expression is a common event in tumorigenesis, and may exacerbate diverse tumorigenic phenotypes by reprogramming post-transcriptional gene regulatory networks.

In certain embodiments, the invention is drawn to methods of using TTP as a biomarker for the diagnosis of cancer. In particular embodiments, lower or repressed levels of TTP compared to non-cancerous tissue correlates to a finding of cancer. For example, if a sample is taken from subject and is determined to be lower or repressed compared to non-cancerous tissue then the subject would be diagnosed with cancer.

In certain embodiments, the invention is drawn to methods of using TTP as a biomarker for the prognosis of cancer. In particular embodiments, lower or repressed levels of TTP correlate to a finding of poor prognosis of cancer or cancer treatment. For example, if a sample is taken from a subject and is determined to be lower or repressed over time in that subject (e.g., by a comparison to an earlier sample taken from the subject) then the subject's prognosis can be determined to be poor, sub-standard, or non-responsive to a particular therapy. Lower or repressed levels of TTP can also correlate to a poor, sub-standard, or non-responsive prognosis as it relates to a decrease in survival time or aggressiveness of the cancer (including, for example, chance of increased cancer metastasis).

In certain embodiments where the invention is drawn to a method of diagnosis, prognosis or other relevant embodiment, an assay or assays are utilized for assessing the quantity of a biological marker, including, for example, TTP, in a sample to determine whether an individual is afflicted with a cancer, whether an individual's pathophysiology associated with a cancer is or has progressed, whether an individual is at risk for (i.e., has a predisposition for or a susceptibility to) developing a cancer, or whether an individual is at risk or experiencing metastatic cancer. A “biological marker” of the instant invention includes, for example, an endogenous molecule that can be measured in vitro, in vivo, ex vivo, or in situ, and that is associated with a cancer.

In certain embodiments, diagnosis or prognosis of a cancer comprises detecting the quantity of TTP nucleotides or polypeptides, and comparing that quantity to a control or other appropriate standard. A control or other appropriate standard is meant to refer to a baseline quantity of TTP that is utilized to determine a diagnosis or prognosis based on the analysis of TTP in a sample from a subject. For example, a baseline quantity of TTP can be obtained from a subject where a sample is obtained, wherein said sample is not affected by a cancer, which such baseline quantity of TTP may serve, for example, as a control or other appropriate standard for the diagnosis of a cancer. Also, for example, a baseline quantity of TTP can be obtained from a subject where a sample is obtained, wherein said sample is affected by a cancer, which such baseline quantity of TTP may serve, for example, as a control or other appropriate standard for the prognosis of a cancer. In particular embodiments, a decrease in the quantity of TTP is important in determining the diagnosis or prognosis of a subject. For example, if a quantity of TTP is decrease compared to a control or other appropriate standard quantity of TTP, wherein the control or other appropriate standard was determined from a sample not affected by a cancer, then a diagnosis of a subject having a cancer is found. Also, for example, if a quantity of TTP is decreased compared to a control or other appropriate standard quantity of TTP, wherein the control or other appropriate standard was determined from a sample affected by a cancer, then a prognosis associated with a pathophysiology of a subject's cancer is found to be progressing or have progressed. Obtaining and using a control or other appropriate standard for the determination of a diagnosis or prognosis of the invention is well know by one of ordinary skill in the art. Therefore, examples recited herein are non-limiting and are meant for illustrative purposes only.

In certain embodiments, a diagnosis or prognosis can be made by analyzing TTP polypeptide, by a variety of methods, including methods described herein, and also generally methods comprising spectroscopy, colorimetry, electrophoresis, isoelectric focusing, immunoprecipitations, and immunofluorescence, and immunoassays (e.g., David et al., U.S. Pat. No. 4,376,110) such as, for example immunoblotting (see also Current Protocols in Molecular Biology, particularly chapter 10). Measuring both quantitative and qualitative decreased of TTP are encompassed by the present invention. For example, in a particular embodiment, an antibody capable of binding to the polypeptide can be used. In a specific embodiment, the antibody comprises a detectable label or the antibody is an antibody that can be detected by a secondary antibody. Antibodies can be polyclonal or monoclonal. An intact antibody, or a fragment thereof (e.g., Fab or F (ab′) 2) can be used. The term “labeled” with regard to the probe or antibody, is intended to encompass direct labeling of the antibody by coupling (i.e., physically linking) a detectable substance to the antibody, as well as indirect labeling of the antibody by reactivity with another reagent that is directly labeled or indirectly labeled. Examples of direct and indirect labels include, for example, a fluorescent moiety, an enzyme, a chromophoric moiety, a radioactive atom, a biotin tag, or a calorimetric tag. Some examples of a fluorescent moiety include rhodamine, fluorescein, TEXAS RED™, etc. Some examples of enzymes include, horseradish peroxidase, glucose oxidase, glucose-6-phosphate dehydrogenase, alkaline phosphatase, beta-galactosidase, urease, luciferase, etc. Some examples of radioactive atoms are ³²P, ¹²⁵I, ³H, etc.

In other certain embodiments diagnosis or prognosis can be made by analyzing TTP nucleic acid, by a variety of methods, including PCR-based methods, DNA array-based, electrophoresis-based methods (including, for example, Southern blot, Northern blot, etc.), and other methods known by those of ordinary skill in the art (see, for example, U.S. Pat. Nos. 4,526,690; 6,232,079; 6,235,504; 6,548,257; 6,830,887; and 6,927,032).

In other certain embodiments, the invention encompasses a kit comprising a reagent or composition as contemplated herein or as would be readily known by one of ordinary skill in the art for the treatment, diagnosis, or prognosis of a cancer. Reagents that are suited for obtaining a sample from an individual may be included in a kit of the invention, such as a syringe, collection vial, needle, or other instruments necessary to take a biopsy or other relevant sample. The kits may comprise, for example, a suitably aliquoted composition and/or additional reagent compositions of the present invention, whether labeled or unlabeled, as may be used to prepare a standard curve for a detection assay. The components of the kit may be packaged in combination or alone in the same or in separate containers, depending on, for example, cross-reactivity or stability, and can also be supplied in solid, liquid, lyophilized, or other applicable form. The container means of the kits will generally include, for example, at least one vial, test tube, flask, bottle, syringe or other container means, into which a component may be placed, and preferably, suitably aliquoted. Where there is more than one component in the kit, the kit can contain a second, third or other additional container into which the additional components may be contained. However, various combinations of components may be comprised in a vial. The kits of the present invention also will typically include a means for containing the composition, additional agent, and any other reagent containers in close confinement for commercial sale. Such containers may include, for example, injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a composition for use in a syringe. In this case, the container means may itself be a syringe, pipette, and/or other such like apparatus, from which the formulation may be applied to an infected area of the body, injected into an animal, and/or even applied to and/or mixed with the other components of the kit. However, in other embodiments the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the composition is placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained. Irrespective of the number and/or type of containers, the kits of the invention may also comprise, and/or be packaged with, an instrument for assisting with the injection/administration and/or placement of a composition within the body of a subject or outside the body of a subject. Such an instrument may be a syringe, pipette, forceps, and/or any such medically approved delivery vehicle.

In certain embodiments, the invention is drawn to modulating the level of TTP for the treatment of cancer. In particular embodiments, the invention is drawn to increasing the cellular level of TTP (including, for example, by means of nucleic acid delivery or protein delivery) for the treatment of cancer.

In certain embodiments of the invention drawn to nucleic acid delivery, the invention encompasses using, for example, a vector (which as used herein refers to a vehicle or other mechanism by which gene delivery or nucleic acid delivery can be accomplished) comprising a gene or nucleic acid contemplated herein (e.g., TTP; see, for example, GenBank Accession No. NM_{—}003407, incorporated by reference) for methods contemplated herein (e.g., for the treatment, diagnosis, or prognosis of cancer). In certain embodiments, gene delivery or nucleic acid delivery can be achieved by a number of mechanisms including, for example, vectors derived from viral and non-viral sources, cation complexes, nanoparticles (including, for example, ormosil and other nano-engineered, organically modified silica, and carbon nanotubes; see for example, Panatarotto et al., Chemistry & Biology. 2003; 10:961-966; Mah et al., Mol. Therapy. 2000; 1:S239; Salata et al., J. Nanobiotechnology. 2004; 2:3) physical methods, bactofection, or any combination thereof.

In certain embodiments, the invention is drawn to gene delivery or nucleic acid delivery comprising the use of viral vectors. Viruses are obligate intra-cellular parasites, designed through the course of evolution to infect cells, often with great specificity to a particular cell type. Viruses tend to be very efficient at transfecting their own DNA into the host cell, which is expressed to produce viral proteins. This characteristic and others, make viruses desirable and viable vectors for gene delivery or nucleic acid delivery. Viral vectors include both replication-competent and replication-defective vectors derived from various viruses. Viral vectors can be derived from a number of viruses, including, for example, polyoma virus, sindbis virus, fowlpox virus (UK 2,211,504 published 5 Jul. 1989), adenovirus and other viruses from the Adenoviridae family, adeno-associated virus and other viruses from the Parvoviridae family, herpes virus, vaccinia virus, alpha-virus, human immunodeficiency virus, papilloma virus, avian virus, cytomegalovirus, retrovirus, hepatitis-B virus, simian virus (including, for example, SV40), and chimeric viruses of any of the foregoing (including, for example, a chimeric adenovirus). Though a number of viral vectors can accomplish gene delivery or nucleic acid delivery, interest has concentrated on a finite number of viral vectors, including, for example, those derived from retrovirus, adenovirus, adeno-associated virus, and herpes virus. Examples of viral vectors include, for example, AAV-MCS (adeno-associated virus), AAV-MCS2 (adeno-associated virus), Ad-Cla (E1/E3 deleted adenovirus), Ad-BGFP-Cla (E1/E3 deleted adenovirus), Ad-TRE (E1/E3 deleted adenovirus), MMP (MPSV/MLV derived retrovirus), MMP-iresGFP (MPSV/MLV derived retrovirus), MMP-iresGFPneo (MPSV/MLV derived retrovirus), SFG-TRE-ECT3 (3′ Enhancer deleted, MLV derived retrovirus), SFG-TRE-IRTECT3 (3′ Enhancer deleted, MLV derived retrovirus), HRST (3′ Enhancer deleted HIV derived retrovirus), simian adenovirus and chimeric adenovirus (see, for example, US Patent Application Publication Nos. 20060211115, 20050069866, 20040241181, 20040171807, 20040136963, and 20030207259).

In other embodiments, gene delivery or nucleic acid delivery also includes vectors comprising polynucleotide complexes comprising cyclodextrin-containing polycations (CDPs), other cationic non-lipid complexes (polyplexes), and cationic lipids complexes (lipoplexes) as carriers for gene delivery or nucleic acid delivery, which condense nucleic acids into complexes suitable for cellular uptake (see, for example, U.S. Pat. No. 6,080,728; Liu et al., Current Medicinal Chemistry, 2003, 10, 1307-1315; Gonazalez et al., Bioconjugate Chemistry 6:1068-1074 (1999); Hwang et al., Bioconjugate Chemistry 12:280-290 (2001)). A systems approach to prepare complexes and modify them with stabilizing and targeting components that result in stable, well-defined DNA- or RNA-containing complexes are suitable for in vivo administration. For example, polycations containing cyclodextrin can achieve high transfection efficiencies while remaining essentially non-toxic. A number of these complexes have been prepared that include variations in charge spacing, charge type, and sugar type (e.g., a spacing of six methylene units between adjacent amidine groups within the co-monomer gave the best transfection properties). Other polyplexes comprise, for example, polyethyleneimime (available from, for example, Avanti Lipids), polylysine (available from, for example, Sigma), polyhistidine (available from, for example, Sigma), and SUPERFECT (available from, for example, Qiagen) (cationic polymer carriers for gene delivery or nucleic acid delivery in vitro and in vivo has been described in the literature, see, for example, Goldman et al., Nature BioTechnology, 15:462 (1997)). Most polyplexes consist of cationic polymers and their complex production is regulated by ionic interactions. One large difference between the methods of action of polyplexes and lipoplexes is that some polyplexes cannot release their polynucleotides into the cytoplasm, which necessitates co-transfection with an endosome-lytic agent (to lyse the endosome that is made during endocytosis, the process by which a polyplex enters the cell) such as, for example, inactivated adenovirus. However this is not always the case, for example, polyplexes comprising polyethylenimine have their own method of endosome disruption, as does chitosan and trimethylchitosan.

Lipoplexes (also known as cationic liposomes) function similar to polyplexes and are complexes comprising positively charged lipids. Lipoplexes are increasingly being used in gene therapy due to their favorable interactions with negatively charged DNA and cell membranes, as well as due to their low toxicity. Due to the positive charge of cationic lipids they naturally complex with the negatively charged DNA. Also as a result of their charge they interact with the cell membrane, endocytosis of a lipoplex occurs and the polynucleotide of interest is released into the cytoplasm. The cationic lipids also protect against degradation of the polynucleotide by the cell. The use of cationic lipids for gene delivery or nucleic acid delivery was initiated by Felgner and colleagues in 1987, who reported that liposomes consisting of N-[1-(2,3-dioleyloxy) propyl]-N,N,N-trimethylammonium chloride (DOTMA) and dioleoylphosphatidylethanolamine (DOPE) were capable of facilitating effective polynucleotide transfer across cell membranes, resulting in high level expression of the encoded gene (Felgner et al., PNAS (1987) 84: 7413-7417). Since this seminal work, many new cationic lipids have been synthesized and have been shown to possess similar transfection activity, many of which are summarized by Balaban et al. (Expert Opinion on Therapeutic Patents (2001), 11(11): 1729-1752).

In other embodiments, gene delivery or nucleic acid delivery of the invention also includes vectors encompassing physical approaches for gene transfer into cells in vitro and in vivo (Gao et al., AAPS Journal. 2007; 9(1): E92-E104). Physical approaches induce transient injuries or defects in cell membranes so that DNA can enter the cells by diffusion. Gene delivery or nucleic acid delivery by physical approaches include, for example, needle injection of naked DNA (see, for example, Wolff et al., Science. 1990; 247:1465-1468), electroporation (see, for example, Heller et al., Expert Opin Drug Deliv. 2005; 2:255-268; Neumann et al., EMBO J. 1982; 1:841-845), gene gun (see, for example, Yang et al., PNAS 1990; 87:9568-9572; Yang et al., Nat. Med. 1995; 1:481-483), ultrasound (see, for example, Lawrie et al., Gene Ther. 2000; 7:2023-2027), hydrodynamic delivery (see, for example, Liu et al., Gene Ther. 1999; 6:1258-1266; Zhang et al., Hum Gene Ther. 1999; 10:1735-1737), and laser-based energy (see, for example, Sagi et al., Prostate Cancer Prostatic Dis. 2003; 6(2):127-30).

In other embodiments, gene delivery or nucleic acid delivery of the invention also includes bactofection (see, for example, Palffy et al., Gene Ther. 2006 January; 13(2):101-5; Loessner et al., Expert Opin Biol Ther. 2004 February; 4(2):157-68; Pilgrim et al., Gene Ther. 2003 November; 10(24):2036-45; Weiss et al., Curr Opin Biotechnol. 2001 October; 12(5):467-72; US Patent Application Publication No. 20030153527; U.S. Pat. Nos. 5,877,159; 6,150,170; 6,500,419; 6,682,729; 7,125,718; 7,393,525). Bacteria-mediated transfer of plasmid DNA into mammalian cells (i.e., bactofection) is a potent approach to introduce a gene or nucleic acid into a large set of different cell types in mammals. Applications include, for example, the expression of a therapeutic protein and RNAi. This mechanism of gene delivery or nucleic acid delivery uses bacteria for the direct transfer of nucleic acids into a target cell or cells. Transformed bacterial strains deliver the genes localized on plasmids into the cells, where these genes or nucleic acids are then expressed. Generally, the method of bactofection comprises using transformed invasive bacteria as a vector to transport genetic material, which is in the form of, for example, a plasmid comprising sequences needed for the transcription and translation of the protein of interest or the delivery of nucleic acids for the purpose of RNAi. For example, bactofection comprises the steps of: (a) transforming invasive bacteria to contain plasmids carrying the transgene; (b) the transformed bacteria penetrates into the cells; (c) vectors are destructed or undergo lysis, which is induced by the presence of the bacteria in the cytoplasm, and release plasmids carried; and (d) the released plasmids get into the nucleus whereupon the transgene is expressed. An analogous series of events transpire in the case of introducing nucleic acids for the purpose of RNAi, expect in that case the nucleic acids decrease or inhibit the expression of one more proteins contemplated by the invention. Bacteria used in bactofection is preferably non-pathogenic or has a minimal pathogenic effect with said bacteria being either naturally occurring or genetically modified and is produced naturally, synthetically, or semi-synthetically. Bactofection has been reported with, for example, species of Shigella, Salmonella, Listeria, and Escherichia coli, with results suggesting that bactofection can be used with any bacterial species (Weiss et al., Curr Opin Biotechnol. 2001 October; 12(5):467-72).

In certain embodiments drawn to protein or amino acid delivery, the invention encompasses using a protein or amino acid sequence contemplated herein (e.g., TTP; see, for example, GenBank Accession No. NP_{—}003398, incorporated by reference) for a method or purpose contemplated herein (e.g., for the treatment, diagnosis, or prognosis of cancer), conjugated to, fused with, or otherwise combined with, a peptide known as a protein transduction domain (“PTP”) for the deliver of the protein or amino acid sequence contemplated herein for the method or purpose contemplated herein. In particular aspects of the invention, TTP (including, for example, conjugated to, fused with, or otherwise combined with a PTD) or a composition comprising TTP is administered to treat cancer. A PTD is a short peptide that facilitates the movement of an amino acid sequence across an intact cellular membrane or barrier, including the blood brain barrier, wherein said amino acid sequence would not penetrate the intact cellular membrane without being conjugated to, fused with, or otherwise combined with a PTD. The conjugation with, fusion to, or otherwise combination of a PTD with a heterologous molecule (including, for example, an amino acid sequence, nucleic acid sequence, or small molecule) is sufficient to cause transduction into a variety of different cells in a concentration-dependent manner. Moreover, when drawn to the delivery of amino acids, it appears to circumvent many problems associated with polypeptide, polynucleotide and drug-based delivery. Without being bound by theory, PTDs are typically cationic in nature causing PTDs to track into lipid raft endosomes and release their cargo into the cytoplasm by disruption of the endosomal vesicle. PTDs have been used for delivery of biologically active molecules, including amino acid sequences (see, for example, Viehl C. T. et al. (2005) Ann. Surg. Oncol. 12:517-525; Noguchi, H., et al. (2004) Nat. Med. 10:305-309 (2004); Fu A. L., et al. (2004) Neurosci. Lett. 368:258-262; Del Gazio Moore et al. (2004) J. Biol. Chem. 279(31):32541-32544; US Application Publication No. 20070105775). For example, it has been shown that TAT-mediated protein transduction can be achieved with large proteins such as beta-galactosidase, horseradish peroxidase, RNAase, and mitochondrial malate dehydrogenase, whereby transduction into cells is achieved by chemically cross-linking the protein of interest to an amino acid sequence of HIV-1 TAT (see, for example, Fawell, S. et al. (1994) Proc. Natl. Acad. Sci. (U.S.A.) 91(2):664-668 (1994); Del Gazio, V. et al. (2003) Mol. Genet. Metab. 80(1-2):170-180 (2003)).

Protein transduction methods encompassed by the invention include an amino acid sequence of the invention conjugated to, fused with, or otherwise combined with, a PTD. In particular embodiments a PTD of the invention includes, for example, the PTD from human transcription factor HPH-1, mouse transcription factor Mph-1, Sim-2, HIV-1 viral protein TAT, Antennapedia protein (Antp) of Drosophila, HSV-1 structural protein Vp22, regulator of G protein signaling R7, MTS, polyarginine, polylysine, short amphipathic peptide carriers Pep-1 or Pep-2, and other PTDs known to one of ordinary skill in the art or readily identifiable to one of ordinary skill in the art (see, for example, US Application Publication No. 20070105775). One of ordinary skill in the art could routinely identify a PTD by, for example, employing known methods in molecular biology to create a fusion protein comprising a potential PTD and, for example, green fluorescent protein (PTD-GFP) and detecting whether or not GFP was able to transduce an intact cellular membrane or barrier, which can be determined by, for example, microscopy and the detection of fluorescence. It is noted that the particular PTD is not limited by any of the foregoing and the invention encompasses any known, routinely identifiable, and after-arising PTD.

Methods of protein transduction are known in the art and are encompassed by the present invention (see, for example, Noguchi, H. et al. (2006) Acta Med. Okayama 60:1-11; Wadia, J. S. et al. (2002) Curr. Opin. Biotechnol. 13:52-56; Viehl C. T. et al. (2005) Ann. Surg. Oncol. 12:517-525; Noguchi, H., et al. (2004) Nat. Med. 10:305-309 (2004); Fu A. L., et al. (2004) Neurosci. Lett. 368:258-262; Del Gazio Moore et al. (2004) J. Biol. Chem. 279(31):32541-32544; US Application Publication No. 2007/0105775; Gump et al. (2007) Trends in Molecular Medicine, 13(10):443-448; Tilstra, J. et al. (2007) Biochem. Soc. Trans. 35(Pt 4):811-815; WO/2006/121579; US Application Publication No. 2006/0222657). In certain embodiments, a PTD may be covalently cross-linked to an amino acid sequence of the invention or synthesized as a fusion protein with an amino acid sequence of the invention followed by administration of the covalently cross-linked amino acid sequence and the PTD or the fusion protein comprising the amino acid sequence and the PTD. In other embodiments, methods for delivering an amino acid sequence of the invention includes a non-covalent peptide-based method using an amphipathic peptide as disclosed by, for example, Morris, M. C. et al. (2001) Nat. Biotechnol. 19:1173-1176 and U.S. Pat. No. 6,841,535; and indirect polyethylenimine cationization as disclosed by, for example, Kitazoe et al. (2005) J. Biochem. 137:693-701.

As a non-limiting illustration of a method of making a PTD fusion protein, an expression system that permits the rapid cloning and expression of in-frame fusion polypeptides using an N-terminal 11 amino acid sequence corresponding to amino acids 47-57 of TAT is used (Becker-Hapak, M. et al. (2001) Methods 24(3):247-56 (2001); Schwarze, F. R. et al. (1999) Science 285:1569-72; Becker-Hapak, M. et al. (2003) Curr. Protoc. Cell Biol. Chapter 20:Unit 20.2). Using this expression system, cDNA of the amino acid sequence of interest is cloned in-frame with an N-terminal 6×His-TAT-HA encoding region in the pTAT-HA expression vector. The 6×His motif provides for the convenient purification of a fusion polypeptide using metal affinity chromatography and the HA epitope tag allows for immunological analysis of the fusion polypeptide. Although recombinant polypeptides can be expressed as soluble proteins using a microorganism (including, for example, E. coli), TAT-fusion polypeptides can often be found within inclusion bodies. In the latter case, these proteins are extracted from purified inclusion bodies in a relatively pure form by lysis in denaturant, such as, for example, 8 M urea. The denaturation aids in the solubilization of the recombinant polypeptide and assists in the unfolding of complex tertiary protein structure which has been observed to lead to an increase in the transduction efficiency over highly-folded, native proteins (Becker-Hapak, M. et al. (2001) Methods 24(3):247-56 (2001)). This latter observation is in keeping with earlier findings that supported a role for protein unfolding in the increased cellular uptake of the TAT-fusion polypeptide TAT-DHFR (Bonifaci, N. et al. (1995) Aids 9:995-1000). It is thought that the higher energy of partial or fully denatured proteins may transduce more efficiently than lower energy, correctly folded proteins, in part due to increased exposure of the TAT domain. Once inside the cells, these denatured proteins are properly folded by cellular chaperones such as, for example, HSP90 (Schneider, C. et al. (1996) Proc. Natl. Acad. Sci. (U.S.A.) 93(25):14536-14541 (1996)). Following solubilization, bacterial lysates are incubated with NiNTA resin (Qiagen), which binds to the 6×His domain in the recombinant protein. After washing, proteins are eluted from the column using increasing concentrations of imidazole. Proteins are further purified using ion exchange chromatography and finally exchanged into PBS+10% glycerol by gel filtration. It is also noted that in certain embodiments where an amino sequence of the invention is conjugated to, fused with, or otherwise combined with a PTD, that such sequences can not only be recombinantly made as described in the specification or as is known by those of ordinary skill in the art, but can also be synthetically or semi-synthetically made as described in the specification or as is known by those of ordinary skill in the art.

In certain embodiments the invention encompasses administration of an amino acid sequence of the invention conjugated to, fused with, or otherwise combined with, a PTD. In other embodiments, the invention encompasses administration of a nucleic acid sequence of the invention conjugated to, fused with, or otherwise combined with, a PTD. Both, an amino acid sequence and a nucleic acid sequence can be transduced across a cellular membrane when conjugated to, fused with, or otherwise combined with, a PTD. As such, administration of an amino acid sequence and a nucleic acid sequence is encompassed by the present invention. Routes of administration of an amino acid sequence or nucleic acid sequence of the invention include, for example, intraarterial administration, epicutaneous administration, ocular administration (e.g., eye drops), intranasal administration, intragastric administration (e.g., gastric tube), intracardiac administration, subcutaneous administration, intraosseous infusion, intrathecal administration, transmucosal administration, epidural administration, insufflation, oral administration (e.g., buccal or sublingual administration), oral ingestion, anal administration, inhalation administration (e.g., via aerosol), intraperitoneal administration, intravenous administration, transdermal administration, intradermal administration, subdermal administration, intramuscular administration, intrauterine administration, vaginal administration, administration into a body cavity, surgical administration (e.g., at the location of a tumor or internal injury), administration into the lumen or parenchyma of an organ, or other topical, enteral, mucosal, or parenteral administration, or other method, or any combination of the forgoing as would be known to one of ordinary skill in the art (see, for example, REMINGTON'S PHARMACEUTICAL SCIENCES, 18th Ed. Mack Printing Company, 1990, incorporated herein by reference).

In certain embodiments where the invention is drawn to methods of using TTP (or other protein or nucleic acid of the invention) to treat or diagnose cancer. “Cancer” refers to, a pathophysiological condition whereby a cell or cells is characterized by dysregulated and/or proliferative cellular growth and the ability to induce said growth, either by direct growth into adjacent tissue through invasion or by growth at distal sites through metastasis, in both, an adult or child, which includes, but is not limited to, carcinomas and sarcomas, such as, for example, acute lymphoblastic leukemia, acute myeloid leukemia, adrenocortical cancer, AIDS-related cancers, AIDS-related lymphoma, anal cancer, astrocytoma (including, for example, cerebellar and cerebral), basal cell carcinoma, bile duct cancer, bladder cancer, bone cancer, brain stem glioma, brain tumor (including, for example, ependymoma, medulloblastoma, supratentorial primitive neuroectodermal, visual pathway and hypothalamic glioma), cerebral astrocytoma/malignant glioma, breast cancer, bronchial adenomas/carcinoids, Burkitt's lymphoma, carcinoid tumor (including, for example, gastrointestinal), carcinoma of unknown primary site, central nervous system lymphoma, cervical cancer, chronic lymphocytic leukemia, chronic myelogenous leukemia, chronic myeloproliferative disorders, colon cancer, colorectal cancer, cutaneous T-Cell lymphoma, endometrial cancer, ependymoma, esophageal cancer, Ewing's Family of tumors, extrahepatic bile duct cancer, eye cancer (including, for example, intraocular melanoma, retinoblastoma, gallbladder cancer, gastric cancer, gastrointestinal carcinoid tumor, gastrointestinal stromal tumor (GIST), germ cell tumor (including, for example, extracranial, extragonadal, ovarian), gestational trophoblastic tumor, glioma, hairy cell leukemia, head and neck cancer, squamous cell head and neck cancer, hepatocellular cancer, Hodgkin's lymphoma, hypopharyngeal cancer, islet cell carcinoma (including, for example, endocrine pancreas), Kaposi's sarcoma, laryngeal cancer, leukemia, lip and oral cavity cancer, liver cancer, lung cancer (including, for example, non-small cell), lymphoma, macroglobulinemia, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myeloma, nasal cavity and paranasal sinus cancer, nasopharyngeal cancer, neuroblastoma, non-Hodgkin's lymphoma, oral cancer, oral cavity cancer, osteosarcoma, oropharyngeal cancer, ovarian cancer (including, for example, ovarian epithelial cancer, germ cell tumor), ovarian low malignant potential tumor, pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, prostate cancer, rectal cancer, retinoblastoma, rhabdomyosarcoma, salivary gland cancer, soft tissue sarcoma, uterine sarcoma, Sézary syndrome, skin cancer (including, for example, non-melanoma or melanoma), small intestine cancer, supratentorial primitive neuroectodermal tumors, T-Cell lymphoma, testicular cancer, throat cancer, thymoma, thymoma and thymic carcinoma, thyroid cancer, transitional cell cancer of the renal pelvis and ureter, trophoblastic tumor (including, for example, gestational), unusual cancers of childhood and adulthood, urethral cancer, endometrial uterine cancer, uterine sarcoma, vaginal cancer, viral induced cancers (including, for example, HPV induced cancer), vulvar cancer, Waldenström's macroglobulinemia, Wilms' Tumor, and women's cancers. In particular embodiments, the cancer is an aggressive cancer of the breast or prostate. An aggressive cancer can be characterized by, for example, the ability to spread from one part of the body to another (i.e., metastasis), the non-responsiveness to treatment (i.e., refractory), decreased survival rates, increased recurrence, etc.

In certain embodiments, the invention is drawn to an mRNA transcript that binds to an ARE-BP and/or a corresponding encoded protein of an mRNA transcript that binds to an ARE-BP as a viable therapeutic option to treat cancer, or for use in the diagnosis or prognosis of cancer. In certain aspects where an mRNA transcript that binds to an ARE-BP (including, or example, TTP) is elevated, as compared to healthy, non-cancerous cells, the invention is drawn to normalizing or restoring the level of such an mRNA transcript. These transcripts include, for example, those shown in Table 1 and other mRNA transcripts recited in the specification or determined by criteria provided herein or known by one of ordinary skill in the art.

TABLE 1
TTP Substrate mRNAs Encoding Pro-Oncogenic Products

| | | % | | | |
|---|---|---|---|---|---|
| | | suppression | | | |
| | Genbank | when TTP | putative TTP binding sites | | |
| mRNA | Accession # | expressed^{a} | sequence^{b} | location^{c} | notes |
| AKT1 | NM_005163 | 79 | uaauuuauu (8/9) | +1648 | Ser/Thr kinase that suppresses |
| | | | | | apoptosis by phosphorylating |
| | | | | | and inactivating components of |
| | | | | | the apoptotic machinery |
| CCND1 | M73554 | 75 | auauuuauu (8/9) | +2310 | cyclin D1 - overexpression |
| (aka bcl-1) | | | uuauuauu (8/9) | +3380 | alters cell cycle progression - |
| | | | uuauuauu (8/9) | +3389 | observed frequently in a variety |
| | | | | | of tumors and may contribute to |
| | | | | | tumorigenesis |
| CDK1 | NM_001798 | 69 | uuauuauu (8/9) | +1234 | essential for cell cycle G1/S |
| | | | uauuuau (7/7) | +1821 | phase transition directing cell |
| | | | | | proliferation |
| ERF-1 | U85658 | 72 | auauuuauu (8/9) | +2098 | transcription factor involved in |
| | | | uuauuuaug (8/9) | +2226 | development - abundance is |
| | | | | | associated with intratubular |
| | | | | | germ cell neoplasias and breast |
| | | | | | cancer |
| NOTCH3 | NM_000435 | 71 | uuauuuaua (8/9) | +7147 | membrane protein that |
| | | | uuauuauu (8/9) | +7290 | establishes an intercellular |
| | | | uuauuauu (8/9) | +7357 | signaling pathway - over- |
| | | | | | expression is associated with |
| | | | | | breast cancer |
| DP-1 | NM_007111 | 65 | uuauuuagu (8/9) | +1452 | cell cycle-regulating |
| | | | | | transcription factor - may have a |
| | | | | | role in hepatocellular carcinoma |
| | | | | | progression by promoting tumor |
| | | | | | cell growth |
| PLAUR | NM_002659 | 45 | uuauuauu (8/9) | +1225 | urokinase plasminogen activator |
| | | | uuauuuauu (9/9) | +1279 | receptor - promotes cell motility |
| | | | | | and metastasis |
| EDN2 | NM_001956 | 62 | uuauuuauu (9/9) | +1098 | endothelin 2 - hypoxia-induced |
| | | | | | autocrine survival factor for |
| | | | | | breast tumor cells that may |
| | | | | | induce macrophage recruitment |
| CYR61 | NM_001554 | 43 | uuauuuauc (8/9) | +1673 | cysteine-rich angiogenic inducer |
| | | | uuauuuaug (8/9) | +1767 | 61 - increased expression is |
| | | | | | associated with an aggressive |
| | | | | | breast cancer cell phenotype |
| CAV2 | NM_001233 | 63 | uauuuau (7/7) | +1167 | caveolin 2 - plasma membrane- |
| | | | | | bound protein associated with |
| | | | | | inflammatory breast cancer |
| VEGF | NM_001025366 | 49 | uuauuuaau (8/9) | +2470 | increases vascular permeability |
| | | | uauuuau (7/7) | +2568 | to induce angiogenesis, also |
| | | | uuauuuauu (9/9) | +2973 | promotes cell migration and |
| | | | uauuuau (7/7) | +3075 | inhibits apoptosis |

mRNA transcripts targeted herein to be normalized or restored to healthy, non-cancerous cell levels can be accomplished by any means including, for example, increased expression of TTP (including by, for example, nucleic acid delivery or protein delivery), small molecule mimics of TTP, or RNA interference (RNAi).

In certain embodiments where RNAi is contemplated, the invention encompasses the use of double-stranded or single-stranded RNA as an interference molecule. RNAi is used to “knock down” or inhibit a particular gene of interest by simply injecting, bathing or feeding to a cell or organism of interest the RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000), although such transfection or recombinant techniques as taught herein and is known by those of ordinary skill in the art can be used to delivery RNAi. It is also noted that RNAi methods are less complex than other types of gene delivery that require expression of the particular nucleic acid of interest.

Another type of RNAi is often referred to as small interfering RNA (siRNA), which may also be utilized for the methods and purposes contemplated herein. A siRNA may comprises a double stranded structure or a single stranded structure, the sequence of which is “substantially identical” to at least a portion of the target gene (See WO 04/046320, which is incorporated herein by reference in its entirety). “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides between such sequences. Identity can be readily calculated (see, for example: Computational Molecular Biology, Lesk, A. M., Oxford University Press, New York, 1988; Biocomputing: Informatics and Genome Projects, Smith, D. W., Academic Press, New York, 1993, and the methods disclosed in WO 99/32619, WO 01/68836, WO 00/44914, and WO 01/36646, all of which are specifically incorporated herein by reference). While a number of methods exist for measuring identity between two nucleotide sequences, the term is well known in the art. Methods for determining identity are typically designed to produce the greatest degree of matching of nucleotide sequence and are also typically embodied in computer programs. Such programs are readily available to those in the relevant art. For example, the GCG program package (Devereux et al), BLASTP, BLASTN, and FASTA (Atschul et al,) and CLUSTAL (Higgins et al., 1992; Thompson, et al., 1994). siRNA methods of the invention contain a nucleotide sequence that is substantially identical to at least a portion of the target gene (see, for example, Table 1) or any other molecular entity associated therewith. One of skill in the art is aware that the nucleic acid sequence a for target gene (see, for example, Table 1) is readily available in GenBank, which is incorporated herein by reference in their entirety. Preferably, the siRNA contains a nucleotide sequence that is completely identical to at least a portion of the target gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. It is preferred that there be 100% sequence identity between the dsRNA for use as siRNA and at least 15 contiguous nucleotides of the target gene (see, for example, Table 1), although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

There are several methods for preparing siRNA, such as chemical synthesis, in vitro transcription, siRNA expression vectors, and PCR expression cassettes. Irrespective of which method one uses, the first step in designing an siRNA molecule is to choose the siRNA target site, which can be any site in the target gene. In certain embodiments, one of skill in the art may manually select the target selecting region of the gene, which may be an ORF (open reading frame) as the target selecting region and may preferably be 50-100 nucleotides downstream of the “ATG” start codon. However, there are several readily available programs available to assist with the design of siRNA molecules, for example siRNA Target Designer by Promega, siRNA Target Finder by GenScript Corp., siRNA Retriever Program by Imgenex Corp., EMBOSS siRNA algorithm, siRNA program by Qiagen, Ambion siRNA predictor, Whitehead siRNA prediction, and Sfold. Thus, it is envisioned that any of the above programs may be utilized in the design and production of siRNA molecules that can be used in the present invention.

In certain embodiments where an mRNA transcript that binds to an ARE-BP is elevated, as compared to healthy, non-cancerous cells, the invention is drawn the diagnosis or prognosis of cancer. These corresponding mRNA transcripts that bind to an ARE-BP include, for example, those shown in Table 1 and other mRNA transcript recited in the specification or determined by criteria provided herein or known by one of ordinary skill in the art. In certain aspects, the elevated level of an mRNA contemplated herein can be quantified and used as a means to diagnose cancer. For example, if one or more of the mRNA transcripts contemplated herein is quantified from a sample taken from a subject and is determined to be elevated then the subject may be diagnosed as having cancer. In another example, if one or more of the mRNA transcripts contemplated herein is quantified from a sample taken from a subject and is determined to be elevated over time in that subject (e.g., by a comparison to an earlier sample taken from the subject) then the subject's prognosis can be determined to be poor (e.g., aggressive cancer), sub-standard, or non-responsive to a particular therapy.

In certain embodiments where a corresponding encoded protein of an mRNA transcript that binds to an ARE-BP is elevated, as compared to healthy, non-cancerous cells, the invention is drawn to normalizing or restoring the level of such an encoded protein to treat cancer. These corresponding encoded proteins of an mRNA transcript that binds to an ARE-BP include, for example, those shown in Table 1 and other encoded protein of an mRNA transcript recited in the specification or determined by criteria provided herein or known by one of ordinary skill in the art. Encoded proteins of an mRNA transcript targeted herein to be normalized or restored to healthy, non-cancerous cell levels can be accomplished by many means including, for example, antibody-based means or other means of interfering with function or binding activity (including, for example, receptors, proteins, and other cellular entities) of the encoded protein including, for example, a truncated protein, peptide, small molecule, etc.

In certain embodiments where a corresponding encoded protein of an mRNA transcript that binds to an ARE-BP is elevated, as compared to healthy, non-cancerous cells, the invention is drawn the diagnosis or prognosis of cancer. These corresponding encoded proteins of an mRNA transcript that binds to an ARE-BP include, for example, those shown in Table 1 and other encoded protein of an mRNA transcript recited in the specification or determined by criteria provided herein or known by one of ordinary skill in the art. In certain aspects, the elevated level of a protein contemplated herein can be quantified and used as a means to diagnose cancer. For example, if one or more of the proteins contemplated herein is quantified from a sample taken from a subject and is determined to be elevated then the subject may be diagnosed as having cancer. In another example, if one or more of the proteins contemplated herein is quantified from a sample taken from a subject and is determined to be elevated over time in that subject (e.g., a comparison to an earlier sample taken from the subject) then the subject's prognosis can be determined to be poor (e.g., aggressive cancer), sub-standard, or non-responsive to a particular therapy.

In certain embodiments of the invention comprising administering an HDAC inhibitor for treating a cancer, an HDAC inhibitor is a molecule that causes a physiological change that stops tumor cells from dividing. In certain aspects, an HDAC inhibitor acts through a mechanism of action whereby the molecule inhibits the activity of histone deacetylase. In certain embodiments, an HDAC inhibitors includes, for example, (i) hydroxamic acids; (ii) cyclic tetrapeptides containing the epoxyketone structure (2S,9S)-2-amino-8-oxo-9,10-epoxy-decanoyl (Aoe); (iii) cyclic peptides not containing Aoe; (iv) benzamides; (v) short-chain and aromatic fatty acids; (vi) derivatives of (i)-(v); (vii) combinations of (i)-(vi); and (viii) miscellaneous compounds. In further certain embodiments, an HDAC inhibitor includes, for example, trichostatin A (TSA), oxamflatin, suberoylanilide hydroxamic acid (SAHA), trapoxin A, trapoxin B. Cγ1-1, Cγ1-2, HC-toxin, WF-3161, chlamydocin, depsipeptide (FK228, formerly known as FR901228), apicidin, sodium butyrate, sodium phenylbutyrate, CHR-3996, CRA-024781, ITF2357, JNJ-26481585, PCI-24781, SB939, JNJ-26854165, pyroxamide, CBHA, trichostatin C, salicylihydroxamic acid (SBHA), azelaic bihydroxamic acid (ABHA), azelaic-1-hydroxamate-9-analide (AAHA), depsipeptide, 6-(3-chlorophenylureido) carpoic hydroxamic acid (3C1-UCHA), A-161906, PXD-101, LAQ-824, MW2796, LBH589, MW2996, Scriptaid (SB-556629), pyroxamide, propenamide, aroyl pyrrolyl hydroxyamide, CI-994, cyclic-hydroxamic-acid-containing peptides (CHAPs), depudecin, tubacin, organosulfur compounds, Pivanex, and MGCD0103, derivatives of any of the foregoing, and combinations of any the foregoing.

In certain embodiments of the invention comprising administering an HDAC inhibitor for treating a cancer wherein elevated levels of TTP demonstrate that the subject is being treated for cancer, elevated levels of TTP is, for example, about a 1 to 50 fold elevation in TTP, a 1 to 40 fold elevation in TTP, 1 to 30 fold elevation in TTP, 1 to 20 fold elevation in TTP, 1 to 10 fold elevation in TTP, 1 to 5 fold elevation in TTP, or 1 to 2.5 fold elevation in TTP. In specific embodiments, elevated levels of TTP is about a 1 fold elevation, about a 2 fold elevation, about a 3 fold elevation, about a 4 fold elevation, about a 5 fold elevation, about a 6 fold elevation, about a 7 fold elevation, about a 8 fold elevation, about a 9 fold elevation, about a 10 fold elevation, about an 11 fold elevation, about a 12 fold elevation, about a 13 fold elevation, about a 14 fold elevation, about a 15 fold elevation, about a 16 fold elevation, about a 17 fold elevation, about a 18 fold elevation, about a 19 fold elevation, about a 20 fold elevation, about a 21 fold elevation, about a 22 fold elevation, about a 23 fold elevation, about a 24 fold elevation, or about a 25 fold elevation.

The present invention also relates to methods of assessing the risk of a subject having cancer, the method comprising determining levels of TTP in a sample suspected of being cancerous in the subject, and comparing the levels of TTP in the sample from the subject to normal levels of TTP. Lower levels of TTP compared to normal levels of TTP would indicate that the subject has an increased risk of having cancer. The methods of assessing risk can be used in any type of cancer including, but not limited to, breast cancer and prostate cancer. Moreover, TTP levels can be assessed on the protein and/or mRNA level.

In one embodiment, the normal levels of TTP are assessed in the same subject from which the sample is taken. In another embodiment, the normal levels are assessed in a sample from the patient that is not suspected of being cancerous. In still another embodiment, the normal levels of TTP are assessed in a population of healthy individuals.

The present invention also relates to methods of increasing the levels of TTP in a cell comprising introducing into the cell a vector, the vector comprising a polynucleotide encoding a TTP protein, the protein comprising residues 2-326 of SEQ ID NO:2. In one embodiment, the vector encodes the full length amino acid sequence of SEQ ID NO:2. In one embodiment, the TTP is part of a fusion protein, such as a fusion between TTP and a trafficking sequence. In another embodiment, the vector comprises the polynucleotide sequence of SEQ ID NO:1.

Another method of increasing the levels of TTP in a cell comprises administering to the cell a histone deacetyltransferase (HDAC) inhibitor and determining the levels of TTP in a cell after administration of the HDAC inhibitor.

In certain embodiments, the methods of increasing expression of TTP in cells, regardless of the methods used, are intended to cause the cell to produce less of at least one pro-oncogenic protein in response to the increased levels of TTP. Examples of pro-oncogenes include, but are not limited to, vascular endothelial cell growth factor A (VEGF-A), caveolin 2 (CAV2), cysteine-rich angiogenic factor 61 (CYR61), endothelin 2 (EDN2), urokinase plasminogen activator receptor (PLAUR), transcription factor DP-1, NOTCH3, transcription factor ERF-1, transcription factor CDK-1, cyclin D1 (CCND1) and Akt1 kinase (AKT1).

While the invention has been described with reference to certain particular embodiments thereof, those skilled in the art will appreciate that various modifications may be made without departing from the spirit and scope of the invention. It is to be expressly understood that any description, figure, example, etc. is provided for the purpose of illustration and description only and is by no means intended to define the limits the invention. Additionally, particular aspects of the invention may not have been reiterated in certain parts of the description, but it will be appreciated by one of ordinary skill in the art that details, descriptions and explanations throughout the specification can be combined even if such details, descriptions and explanations are not laid out in contiguous form.

All patents and publications mentioned in this specification are indicative of the level of those skilled in the art to which the invention pertains. All patents and publications cited herein are incorporated by reference to the same extent as if each individual publication was specifically and individually indicated as having been incorporated by reference in its entirety.

### EXAMPLES

### Example 1

HeLa/Tet-Off cells (Clontech) were maintained at 37° C. and 5% CO₂ in DMEM (Invitrogen) supplemented with 10% FCS (Atlanta Biologicals) and 100 μg/mL G148 (Cellgro). Plasmids pT2hyg-FLAG-TTPwt and pT2hyg-FLAG-TTP C147R were transfected using Superfect (Qiagen), and stably transformed HeLa cell clones were isolated by selection in 100 μg/mL hygromycin B (Roche). Doxycycline (Sigma) was maintained (2 μg/mL) during selection and subsequent clonal expansion to prevent any tristetraprolin-dependent effects on cell viability. Several dozen independent hygromycin-resistant lines were screened for doxycycline-regulated expression of FLAG-TTPwt (or C 147R) by Western blot using anti-FLAG antibodies.

HeLa or MEF cells were seeded in 96-well plates at 1,000 per well and then returned to the tissue culture incubator. When measuring proliferation rates, cells were counted using the DHL Cell Viability and Proliferation Assay Kit (Anaspec) according to the manufacturer's instructions. Cell numbers were determined by comparison of background-corrected fluorescence to standard curves of fluorescence versus cell number for each cell type and were consistent with data obtained from trypan blue exclusion assays (data not shown). To measure the sensitivity of HeLa and MEF lines to proapoptotic stimuli, cells were similarly seeded in 96-well plates and allowed to grow for 24 h before adding varying concentrations of staurosporine or cisplatin. Twenty-four hours afterwards, surviving cells were counted as described above. The IC₅₀ for each apoptotic stimulus was resolved using a four-parameter logistic equation (PRISM version 3.03).

Murine embryonic fibroblast (MEF) cultures were derived from E14.5 embryos of tristetraprolin knockout mice (Zfp36^{-/-}) and wild-type littermates (Zfp36^{+/+}) as described in previous publications and were maintained in DMEM supplemented with 10% FCS, 100 units/mL penicillin, 100 μg/mL streptomycin, and 2 mmol/L L-glutamine (Cellgro). All experiments involving MEFs in this study were done before the 12th cell passage.

### Example 2

Cellular VEGF mRNA decay rates were measured using actinomycin D time-course assays. Briefly, transcription was inhibited by addition of actinomycin D (5 μg/mL; Calbiochem) to the culture medium, and total RNA was purified at selected times thereafter. Time courses were limited to 4 h to avoid complicating cellular mRNA decay pathways by actinomycin D-enhanced apoptosis. VEGF mRNA levels were measured at each time point by quantitative real-time reverse transcription-PCR (RT-PCR) and normalized to glyceraldehyde-3-phosphate dehydrogenase mRNA. First-order decay constants (k) were solved by nonlinear regression (PRISM) of the percentage of VEGF mRNA remaining versus time of actinomycin D treatment. Resolved VEGF mRNA half-lives (t½=ln 2/k) are based on the mean±SD of n independent time course experiments where n 3 or the mean±spread where n=2. Ribonucleoprotein immunoprecipitations used to detect interactions between FLAG-TTP and cellular VEGF mRNA were adapted from previously described methods.

### Example 3

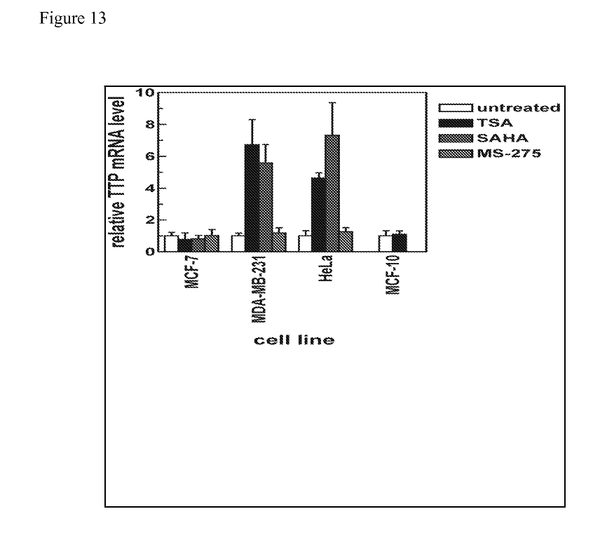

Comparisons of TTP expression between MCF-7 and MDA-MB-231 cells revealed that transcription of the TTP gene is specifically repressed in the MDA cell model. To test whether hypoacetylation of the TTP gene contributed to suppression of its expression in MDA-MB-231 cells, TTP mRNA levels were measured in cells before and after treatment with a selection of HDAC inhibitors. Of the selected compounds, no change in TTP expression was observed in minimally tumorigenic MCF-7 cells or non-tumorigenic MCF-10A cells (FIG. 13). By contrast, the broad spectrum HDAC inhibitor trichostatin A (TSA) potently induced TTP mRNA levels in two aggressive cancer cell models: MDA-MB-231 and the cervical adenocarcinoma cell line HeLa. Furthermore, TTP mRNA was induced in these cell models by treatment with suberoylanilide hydroxamic acid (SAHA; also known as Vorinostat), an inhibitor of class I and TI HDACs that has been approved for treatment of cutaneous T-cell lymphoma by the FDA. Notably, TTP expression was not activated in these cell models by MS-275 (also known as Entinostat), which preferentially inhibits HDAC 1. Together, these data indicate that transcriptional suppression of TTP observed in aggressive cancer cell models can be alleviated by a subset of HDAC inhibitors, demonstrating that expression of TTP in tumors is useful for determining which patients would derive maximal benefit from HDAC inhibitor therapy (i.e., TTP can be used for determining responders to HDAC inhibitor therapy).

What is claimed is:

1. A method of determining if an anti-cancer treatment will effectively treat a cancerous tissue, the method comprising
a) determining levels of tristetraprolin (TTP) in the cancerous tissue, and
b) comparing the levels of TTP in the cancerous tissue to normal levels of TTP
wherein low levels of TTP in the cancerous tissue compared to normal levels of TTP indicate that the anti-cancer therapy may be effective in treating the cancerous tissue.

2. The method of claim 1, wherein the normal levels of TTP are assessed in the same subject from which the cancerous tissue is taken.

3. The method of claim 1, wherein the normal levels are assessed in a sample from a subject that has not been diagnosed with cancer.

4. The method of claim 1, wherein the normal levels of TTP are assessed in a population of healthy individuals.

5. The method of claim 1, wherein the cancerous tissue is breast cancer or prostate cancer.

6. The method of claim 5, wherein the anti-cancer therapy comprises at least one histone deacetyltransferase (HDAC) inhibitor.

7. The method of claim 6, wherein the HDAC inhibitor is a compound selected from the group consisting of trichostatin A (TSA) and suberoylanilide hydroxamic acid (SAHA).

8. The method of claim 1, wherein the TTP levels are determined by measuring levels of mRNA transcripts that code for TTP protein.

9. The method of claim 1, wherein the TTP levels are determined by measuring TTP protein levels.

10. A method of assessing the progression of cancer in a subject having cancer, the method comprising
a) determining levels of tristetraprolin (TTP) in a cancerous sample in the subject at a first and second time point, and
b) comparing the levels of TTP from the first and second time points to determine a change in the levels of TTP over time,
wherein increased levels of TTP over time indicates that the cancer in the subject may be regressing, and wherein decreased levels of TTP over time indicates that the cancer in the subject may be progressing.

11. The method of claim 10, wherein the cancer is selected from the group consisting of breast cancer and prostate cancer.

12. The method of claim 10, wherein the TTP levels are determined by measuring levels mRNA transcripts that code for TTP protein.

13. The method of claim 10, wherein the TTP levels are determined by measuring TTP protein levels.

14. The method of claim 10, wherein the subject receives an anti-cancer therapy prior to the first time point.

15. The method of claim 10, wherein said the subject receives an anti-cancer therapy after the first time point.

16. The method of claim 10, wherein determining levels of tristetraprolin (TTP) in a cancerous sample in the subject at a first time and second time point comprises administering at least one histone deacetyltransferase (HDAC) inhibitor at a time point after said first time point and before said second time point, wherein increased levels of TTP over time indicates that the cancer in the subject may be regressing, and wherein decreased levels of TTP over time indicates that the cancer in the subject may be progressing.

17. The method of claim 11, wherein the HDAC inhibitor is a compound selected from the group consisting of trichostatin A (TSA) and suberoylanilide hydroxamic acid (SAHA).

18. A kit for assessing levels of tristetraprolin (TTP) in a sample, the kit comprising an a binding entity selected from the group consisting of antibody directed towards an epitope of TTP, an antibody fragment directed towards an epitope of TTP and a polynucleotide that is complementary to a portion of an mRNA transcript that codes for TTP protein.

19. The kit of claim 18, wherein the binding entity is an intact antibody.

20. The kit of claim 18, wherein the binding entity is a polynucleotide that is complementary to a portion of an mRNA transcript that codes for TTP protein.
